(19) United States
(12) Patent Application Publication
(10) Pub. No.: US 2025/0188418 A1
(43) Pub. Date: Jun. 12, 2025

(54) USE OF MIR-22-3P IN PROMOTING DIFFERENTIATION OF YAK MYOBLASTS

(71) Applicants: Lanzhou Institute of Husbandry and Pharmaceutical Sciences, Chinese Academy of Agricultural Sciences, Lanzhou (CN); Institute of Western Agriculture, the, Chinese Academy of Agricultural Sciences, Changji City (CN)

(72) Inventors: Xiaoming MA, Lanzhou (CN); Chunnian LIANG, Lanzhou (CN); Ping YAN, Lanzhou (CN); Xian GUO, Lanzhou (CN); Min CHU, Lanzhou (CN); Yongfu LA, Lanzhou (CN); Pengjia BAO, Lanzhou (CN); Xiaoyun WU, Lanzhou (CN); Tong Wang, Lanzhou (CN); Qianyun GE, Lanzhou (CN)

(73) Assignees: Lanzhou Institute of Husbandry and Pharmaceutical Sciences, Chinese Academy of Agricultural Sciences, Lanzhou (CN); Institute of Western Agriculture, the, Chinese Academy of Agricultural Sciences, Lanzhou (CN)

(21) Appl. No.: 18/972,626

(22) Filed: Dec. 6, 2024

(30) Foreign Application Priority Data
Dec. 6, 2023 (CN) 2023116587452

(51) Int. Cl. C12N 5/077

(52) U.S. Cl. CPC C12N 5/0658; C12N 2501/65

(57) ABSTRACT

The present disclosure relates to the technical field of gene engineering, and in particular, to use of miR-22-3p in promoting the differentiation of yak myoblasts. miR-22-3pmimic can significantly promote the expression of miR-22-3p, and can also significantly increase the expression of differentiation marker genes MYF5 and MYOD at the mRNA and protein levels. miR-22-3p inhibitor can significantly inhibit the expression of miR-22-3p, and can significantly promote the expression of MYF5 and MYOD at the mRNA and protein levels. miR-22-3p mimic can significantly promote the formation of myotubes, while miR-22-3p inhibitor can significantly inhibit the formation of myotubes. The above results indicate that miR-22-3p has a promoting effect in the differentiation process of myoblasts.

### INCORPORATION BY REFERENCE

The instant application contains a Sequence Listing which has been submitted electronically in XML format and is hereby incorporated by reference in its entirety. The name of the text file containing the sequence listing is SEQUENCE.xml, has a file size of 10,414 bytes, and was created on Dec. 6, 2024.

This application claims the benefit of priority from China Patent Application No. 2023116587452 filed on Dec. 6 2023, the contents of which are hereby incorporated by reference in their entirety.

### TECHNICAL FIELD

The present disclosure relates to the technical field of gene engineering, and particularly to use of miR-22-3p in promoting differentiation of yak myoblasts.

### BACKGROUND

The production traits of yaks are mainly manifested in the growth and development process of skeletal muscle, and the growth rate and quantity of skeletal muscle directly affect the yield of yak meat. The development of skeletal muscle is a complex physiological process that includes an increase in the number of fibers (before birth) and an increase in fiber volume (after birth). In the postnatal stage, animals achieve skeletal muscle growth by increasing the volume of muscle fibers, which mainly depends on the proliferation, differentiation and protein turnover of myoblasts. Myoblasts are a special type of myogenic stem cells that participate in two biological processes: the first function is regeneration, when mature muscle fiber cells are damaged, myoblasts will be activated, then proliferate, migrate and differentiate to form new myocytes, and eventually fuse with other cells to form muscle fibers; the second function is to maintain the number of muscle fiber nuclei, thereby ensuring a relatively balanced ratio of muscle fiber nuclei and cytoplasm.

Numerous studies have shown that miRNA affects the growth metabolism of skeletal muscle by regulating transcription factors and signaling mechanisms. Skeletal muscle satellite cells play a key role in the formation and regeneration of skeletal muscle. In skeletal muscle, there are a large number of expressed miRNAs, known as MyomiRs. Among them, miR-1, miR-206 and miR-133 are the main members of MyomiRs. Among these miRNAs, miR-1 and miR-133 are specifically expressed in skeletal muscle and cardiac muscle, while miR-206 is primarily expressed in skeletal muscle. These miRNAs play different roles in the proliferation and differentiation of skeletal muscle. For example, miR-1 is involved in cardiac hypertrophy, and miR-133a is involved in cardiomyocyte proliferation and inhibits the expression of smooth muscle genes.

In addition to muscle-specific miRNAs, non-muscle-specific miRNAs, such as miR-143 and miR-145, also play an important role in regulating smooth muscle cell fate and regeneration. These research findings strongly demonstrate the importance of miRNAs in the growth and differentiation of skeletal muscle. However, there is no report in the prior art on whether miR-22-3p can promote the differentiation of yak myoblasts.

### SUMMARY

In order to solve the above problems, the present disclosure provides use of miR-22-3p in promoting differentiation of yak myoblasts, which proves that miR-22-3p has the effect of promoting the differentiation of yak myoblasts.

In order to achieve the above object, the present disclosure provides the following technical solutions:

The present disclosure provides use of miR-22-3p in promoting differentiation of yak myoblasts.

The present disclosure also provides use of miR-22-3p in promoting formation of yak myotubes.

The present disclosure has the following beneficial effects:

According to the present disclosure, miR-22-3p mimic and miR-22-3p inhibitor are used to transfect myoblasts respectively. After transfection, cells are induced to differentiate, and cells are collected for detection of mRNA and protein levels 24 hours after induction of differentiation. QPCR and Westernblot results show that miR-22-3pmimic significantly promotes the expression of miR-22-3p and significantly increases the expression of differentiation marker genes MYF5 and MYOG at the mRNA and protein levels; miR-22-3p inhibitor significantly inhibits the expression of miR-22-3p and significantly promotes the expression of MYF5 and MYOG at the mRNA and protein levels. In the present disclosure, immunofluorescence experiments are carried out to detect the effects of miR-22-3pmimic and inhibitor on myotube formation. The results show that miR-22-3p mimic significantly promotes the formation of myotubes, while miR-22-3p inhibitor significantly inhibits the formation of myotubes. The above results indicate that miR-22-3p has a promoting effect in the differentiation process of myoblasts.

### BRIEF DESCRIPTION OF DRAWINGS

In order to illustrate the embodiments of the present disclosure or the technical solutions in the prior art more clearly, the accompanying drawings used in the embodiments are briefly described below.

FIG. 1 is a schematic diagram of a dual fluorescent vector plasmid pmirGLO;

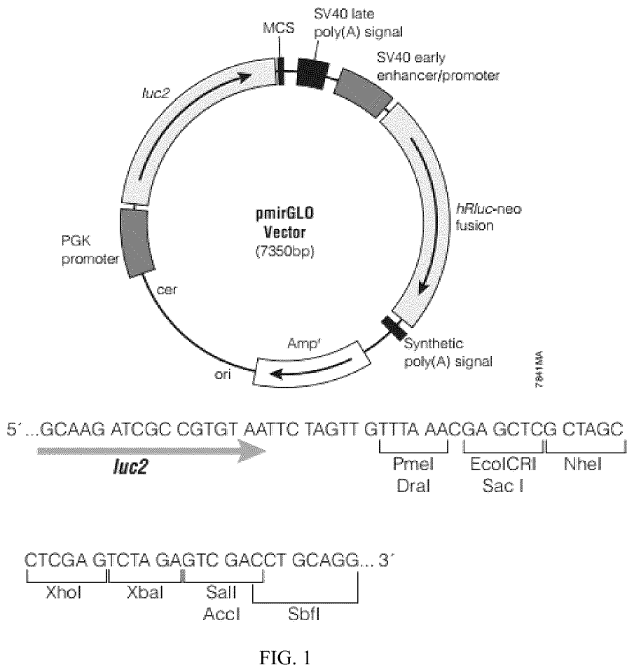

FIG. 2 shows a binding site of miR-22-3p and MEG8/RTL1;

FIG. 3 shows a dual-luciferase experiment verifying the targeting regulatory relationships of MEG8/miR-22-3p and RTL1/miR-22-3p;

FIG. 4 shows the expression level of miR-22-3p at different differentiation stages of myoblasts;

FIG. 5 shows that miR-22-3p mimic promotes myoblast differentiation;

FIG. 6 shows that miR-22-3p inhibitor inhibits myoblast differentiation;

FIG. 7 shows that RTL1-OE inhibits myoblast differentiation;

FIG. 8 shows that RTL1-Si promotes myoblast differentiation;

FIG. 9 shows the regulatory relationships of MEG8-RTL1-miR-22-3p.

### DETAILED DESCRIPTION OF EMBODIMENTS

The present disclosure provides use of miR-22-3p in promoting differentiation of yak myoblasts.

The present disclosure further provides use of miR-22-3p in promoting formation of yak myotubes.

In order to further illustrate the present disclosure, the present disclosure is described in detail below in conjunction with embodiments, but these embodiments should not be construed as limiting the scope of protection of the present disclosure.

### Example 1

### 1. Experimental Method

### 1.1 Construction of Adenovirus-Mediated RTL1 Overexpression Vector

In this example, AdEasy adenovirus expression vector system was used. General Biol (Anhui) Co., Ltd. was entrusted to synthesize the full-length CDS (cording sequence) of the RTL1 gene by using the bovine RTL1 gene mRNA sequence (NM_095020.1) published in GenBank as a template. The CDS sequence was digested with BglII/HindIII restriction endonucleases and ligated into pShuttle-CMV (purchased from Fenghui Biotechnology, Cat. No. BR009) shuttle vector, and pShuttle-CMV-RTL1 was homologously recombined with pAdEasy-1 backbone vector in BJ5183 competent cells. The recombinant plasmid was digested with PacI restriction endonuclease and linearized, and then transfected into Hek-293A cells for adenovirus packaging and propagation. In this example, the RTL1 recombinant overexpression adenovirus was named RTL1-OE. The negative control (NC) virus used in this example was routinely purchased and was named OE-NC in this example. The expression efficiency of adenovirus-mediated RTL1 overexpression vector was identified by RT-PCR and Westernblot methods, respectively.

### 1.2 Construction of Adenovirus-Mediated RNA Interference Vector

In this example, to construct an adenovirus-mediated RNA interference vector, the interference target of RTL1 gene was designed using the bovine RTL1 gene mRNA sequence as a template, and General Biol (Anhui) Co., Ltd. was entrusted to synthesize the interference sequence (SEQ IDNo. 1) ACTAAAGATATTTGCATGTCGCTATGTGTTCTGGGAAATCACCATAA ACGTG AAATGTCTTTGGATTTGGGAATCTTATAAGTTCTGTATGAGA CCACTCAGAC GATGATGGAGCGTAAGAttcaagagaTCTTACGCTCCATC ATCGTCTttttt, and meanwhile shRNA was ligated into pShuttle-CMV vector by BglII/SalI digestion. In BJ5183 competent cells, pShuttle-CMV-shRTL1 was homologously recombined with pAdEasy-1 backbone vector. The recombinant plasmid was extracted and linearized by PacI restriction endonuclease digestion, and then transfected into Hek-293A cells for adenovirus packaging and propagation. In this example, the recombinant shRNA interference adenovirus and the control adenovirus were named sh-RTL1 and sh-NC, respectively. The interference efficiency of the adenovirus-mediated RNA interference vector was identified by RT-PCR method and Westernblot method respectively.

1.3 Construction of miR-22-3p Lentiviral Vector

To construct a miRNA overexpression/interference lentiviral vector, General Biol (Anhui) Co., Ltd. was entrusted to synthesize the interference or overexpression sequence and ligate the sequence to the lentiviral vector pLVX-shRNA1. After the ligation was completed, the recombinant vector was transformed into E. coli for amplification and extraction, and sequencing was performed to confirm that the inserted sequence had been successfully ligated into the lentiviral vector. The verified vector was transfected into the HEK293T cell line. The Helper vectors could assist in packaging and amplification of the lentivirus. After packaging, the lentiviral fluid was collected and used for further miRNA interference or overexpression experiments. During the experiment, appropriate experimental conditions and control groups must be used to verify the effect of miRNA interference or overexpression, and further functional studies were conducted.

1.4 Construction of Dual-Luciferase Reporter Gene Vector for miR-22-3p/MEG8 Binding Site and RTL1/miR-22-3p Binding Site

The RTL1/miR-22-3p and miR-22-3p/MEG8 binding sites were predicted using RNAhybrid and Starbase. PCR amplification was performed according to the binding site sequences in the NCBI database. Meanwhile, the binding site mutation fragments were amplified, and the amplified fragments were recovered and ligated into the pmirGLO vector. After successful ligation, an endotoxin-free plasmid extraction kit was used for plasmid extraction.

The nucleotide sequence of the dual-luciferase reporter gene vector is shown in SRQ ID No. 2:

CATGCAAGCTGATCCGGCTGCTAACAAAGCCCGAAAGGAAGC
TGAGTTGGCTGCTGCCACCGCTGAGCAATAACTAGCATAACCCCTTG
GGGCGGCCGCTTCGAGCAGACATGATAAGATACATTGATGAGTTTGG
ACAAACCACAACTAGAATGCAGTGAAAAAAATGCTTTATTTGTGAAA
TTTGTGATGCTATTGCTTTATTTGTAACCATTATAAGCTGCAATAAAC
AAGTTAACAACAACAATTGCATTCATTTTATGTTTCAGGTTCAGGGG
GAGATGTGGGAGGTTTTTTTAAGCAAGTAAAACCTCTACAAATGTGG
TAAAATCGAATTTTAACAAAATATTAACGCTTACAATTTCCTGATGC
GGTATTTTCTCCTTACGCATCTGTGCGGTATTTCACACCGCATACGCG
GATCTGCGCAGCACCATGGCCTGAAATAACCTCTGAAAGAGGAACTT
GGTTAGGTACCTTCTGAGGCGGAAAGAACCAGCTGTGGAATGTGTGT
CAGTTAGGGTGTGGAAAGTCCCCAGGCTCCCCAGCAGGCAGAAGTAT
GCAAAGCATGCATCTCAATTAGTCAGCAACCAGGTGTGGAAAGTCCC
CAGGCTCCCCAGCAGGCAGAAGTATGCAAAGCATGCATCTCAATTAG
TCAGCAACCATAGTCCCGCCCCTAACTCCGCCCATCCCGCCCCTAACT
CCGCCCAGTTCCGCCCATTCTCCGCCCCATGGCTGACTAATTTTTTTT
ATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTTG
ATTCTTCTGACACAACAGTCTCGAACCAAAGGCTGGAGCCACCATGG
CTTCCAAGGTGTACGACCCCGAGCAACGCAAACGCATGATCACTGGG
CCTCAGTGGTGGGCTCGCTGCAAGCAAATGAACGTGCTGGACTCCTT
CATCAACTACTATGATTCCGAGAAGCACGCCGAGAACGCCGTGATTT
TTCTGCATGGTAACGCTGCCTCCAGCTACCTGTGGAGGCACGTCGTG
CCTCACATCGAGCCCGTGGCTAGATGCATCATCCCT
GATCTGATCGGAATGGGTAAGTCCGGCAAGAGCGGGAATGGC
TCATATCGC
CTCCTGGATCACTACAAGTACCTCACCGCTTGGTTCGAGCTGC
TGAACCTT
CCAAAGAAAATCATCTTTGTGGGCCACGACTGGGGGGCTTGTC
TGGCCTTT
CACTACTCCTACGAGCACCAAGACAAGATCAAGGCCATCGTC
CATGCTGA
GAGTGTCGTGGACGTGATCGAGTCCTGGGACGAGTGGCCTGA
CATCGAGG
AGGATATCGCCCTGATCAAGAGCGAAGAGGGCGAGAAAATGG
TGCTTGAG
AATAACTTCTTCGTCGAGACCATGCTCCCAAGCAAGATCATGC
GGAAACTG
GAGCCTGAGGAGTTCGCTGCCTACCTGGAGCCATTCAAGGAG
AAGGGCGA
GGTTAGACGGCCTACCCTCTCCTGGCCTCGCGAGATCCCTCTC
GTTAAGGG
AGGCAAGCCCGACGTCGTCCAGATTGTCCGCAACTACAACGC
CTACCTTC
GGGCCAGCGACGATCTGCCTAAGATGTTCATCGAGTCCGACCC
TGGGTTCT
TTTCCAACGCTATTGTCGAGGGAGCTAAGAAGTTCCCTAACAC
CGAGTTCG
TGAAGGTGAAGGGCCTCCACTTCAGCCAGGAGGACGCTCCAG
ATGAAATG
GGTAAGTACATCAAGAGCTTCGTGGAGCGCGTGCTGAAGAAC
GAGCAGAC
CGGTGGTGGGAGCGGAGGTGGCGGATCAGGTGGCGGAGGCTC
CGGAGGG
ATTGAACAAGATGGATTGCACGCAGGTTCTCCGGCCGCTTGGG
TGGAGAG
GCTATTCGGCTATGACTGGGCACAACAGACAATCGGCTGCTCT
GATGCCGC
CGTGTTCCGGCTGTCAGCGCAGGGGCGCCCGGTTCTTTTTGTC
AAGACCG
ACCTGTCCGGTGCCCTGAATGAACTGCAGGACGAGGCAGCGC
GGCTATCG
TGGCTGGCCACGACGGGCGTTCCTTGCGCAGCTGTGCTCGACG
TTGTCAC
TGAAGCGGGAAGGGACTGGCTGCTATTGGGCGAAGTGCCGGG
GCAGGATC
TCCTGTCATCTCACCTTGCTCCTGCCGAGAAAGTATCCATCAT
GGCTGATGC
AATGCGGCGGCTGCATACGCTTGATCCGGCTACCTGCCCATTC
GACCACCA
AGCGAAACATCGCATCGAGCGAGCACGTACTCGGATGGAAGC
CGGTCTTG
TCGATCAGGATGATCTGGACGAAGAGCATCAGGGGCTCGCGC
CAGCCGAA
CTGTTCGCCAGGCTCAAGGCGCGCATGCCCGACGGCGAGGAT
CTCGTCGT
GACCCATGGCGATGCCTGCTTGCCGAATATCATGGTGGAAAAT
GGCCGCTT
TTCTGGATTCATCGACTGTGGCCGGCTGGGTGTGGCGGACCGC
TATCAGGA
CATAGCGTTGGCTACCCGTGATATTGCTGAAGAGCTTGGCGGC
GAATGGGC
TGACCGCTTCCTCGTGCTTTACGGTATCGCCGCTCCCGATTCGC
AGCGCATC
GCCTTCTATCGCCTTCTTGACGAGTTCTTCTGAGCGGGACTCTG
GGGTTCG
AAATGACCGACCAAGCGACGCCCAACCTGCCATCACGATGGC
CGCAATAA
AATATCTTTATTTTCATTACATCTGTGTGTTGGTTTTTTGTGTG
AATCGATAG
CGATAAGGATCCTCTTTGCGCTTGCGTTTTCCCTTGTCCAGATA
GCCCAGTA
GCTGACATTCATCCGGGGTCAGCACCGTTTCTGCGGACTGGCT
TTCTACGT
AATGGTTTCTTAGACGTCAGGTGGCACTTTTCGGGGAAATGTG
CGCGGAA
CCCCTATTTGTTTATTTTTCTAAATACATTCAAATATGTATCCG
CTCATGAGA
CAATAACCCTGATAAATGCTTCAATAATATTGAAAAAGGAAG
AGTATGAGTA
TTCAACATTTCCGTGTCGCCCTTATTCCCTTTTTTGCGGCATTTT
GCCTTCCT
GTTTTTGCTCACCCAGAAACGCTGGTGAAAGTAAAAGATGCTG
AAGATCA
GTTGGGTGCACGAGTGGGTTACATCGAACTGGATCTCAACAGC
GGTAAGA
TCCTTGAGAGTTTTCGCCCCGAAGAACGTTTTCCAATGATGAG
CACTTTCA
AAGTTCTGCTATGTGGCGCGGTATTATCCCGTATTGACGCCGG
GCAAGAGC
AACTCGGTCGCCGCATACACTATTCTCAGAATGACTTGGTTGA
GTACTCAC
CAGTCACAGAAAAGCATCTTACGGATGGCATGACAGTAAGAG
AATTATGC
AGTGCTGCCATAACCATGAGTGATAACACTGCGGCCAACTTAC
TTCTGACA
ACTATCGGAGGACCGAAGGAGCTAACCGCTTTTTTGCACAACA
TGGGGGA
TCATGTAACTCGCCTTGATCGTTGGGAACCGGAGCTGAATGAA
GCCATACC
AAACGACGAGCGTGACACCACGATGCCTGTAGCAATGGCAAC
AACGTTGC
GCAAACTATTAACTGGCGAACTACTTACTCTAGCTTCCCGGCA
ACAATTAAT
AGACTGGATGGAGGCGGATAAAGTTGCAGGACCACTTCTGCG
CTCGGCCC
TTCCGGCTGGCTGGTTTATTGCTGATAAATCTGGAGCCGGTGA
GCGTGGGT
CTCGCGGTATCATTGCAGCACTGGGGCCAGATGGTAAGCCCTC
CCGTATCG
TAGTTATCTACACGACGGGGAGTCAGGCAACTATGGATGAAC
GAAATAGAC
AGATCGCTGAGATAGGTGCCTCACTGATTAAGCATTGGTAATT
CGAAATGA
CCGACCAAGCGACGCCCAACCGGTATCAGCTCACTCAAAGGC
GGTAATAC
GGTTATCCACAGAATCAGGGGATAACGCAGGAAAGAACATGT
GAGCAAAA
GGCCAGCAAAAGGCCAGGAACCGTAAAAAGGCCGCGTTGCTG
GCGTTTTT
CCATAGGCTCCGCCCCCCTGACGAGCATCACAAAAATCGACG
CTCAAGTC
AGAGGTGGCGAAACCCGACAGGACTATAAAGATACCAGGCGT
TTCCCCCT
GGAAGCTCCCTCGTGCGCTCTCCTGTTCCGACCCTGCCGCTTA
CCGGATAC
CTGTCCGCCTTTCTCCCTTCGGGAAGCGTGGCGCTTTCTCATAG
CTCACGC
TGTAGGTATCTCAGTTCGGTGTAGGTCGTTCGCTCCAAGCTGG
GCTGTGTG
CACGAACCCCCCGTTCAGCCCGACCGCTGCGCCTTATCCGGTA
ACTATCGT
CTTGAGTCCAACCCGGTAAGACACGACTTATCGCCACTGGCAG
CAGCCAC
TGGTAACAGGATTAGCAGAGCGAGGTATGTAGGCGGTGCTA
CAGAGTTCTT
GAAGTGGTGGCCTAACTACGGCTACACTAGAAGGACAGTATT
TGGTATCTG
CGCTCTGCTGAAGCCAGTTACCTTCGGAAAAAGAGTTGGTAG
CTCTTGATC
CGGCAAACAAACCACCGCTGGTAGCGGTGGTTTTTTTGTTTG
CAAGCAGC
AGATTACGCGCAGAAAAAAAGGATTTCAAGAAGATCCTTTG
ATCTTTTCTA
CGGGGTCTGACGCTCAGTGGAACGAAAACTCACGTTAAGGG
ATTTTGGTC
ATGAGATTATCAAAAAGGATCTTCACCTAGATCCTTTTATAG
TCCGGAAATA
CAGGAACGCACGCTGGATGGCCCTTCGCTGGGATGGTGAAAC
CATGAAAA
ATGGCAGCTTCAGTGGATTAAGTGGGGGTAATGTGGCCTGTA
CCCTCTGGT
TGCATAGGTATTCATACGGTTAAAATTTATCAGGCGCGATTG
CGGCAGTTTT
TCGGGTGGTTTGTTGCCATTTTTACCTGTCTGCTGCCGTGATC
GCGCTGAAC
GCGTTTTAGCGGTGCGTACAATTAAGGGATTATGGTAAATCC
ACTTACTGTC
TGCCCTCGTAGCCATCGAGATAAACCGCAGTACTCCGGCCAC
GATGCGTCC
GGCGTAGAGGATCGAGATCTACCGGGTAGGGGAGGCGCTTTT
CCCAAGGC
AGTCTGGAGCATGCGCTTTAGCAGCCCCGCTGGGCACTTGGC
GCTACACA
AGTGGCCTCTGGCCTCGCACACATTCCACATCCACCGGTAGG
CGCCAACC
GGCTCCGTTCTTTGGTGGCCCCTTCGCGCCACCTTCTACTCCT
CCCCTAGTC
AGGAAGTTCCCCCCCGCCCCGCAGCTCGCGTCGTGCAGGACG
TGACAAAT
GGAAGTAGCACGTCTCACTAGTCTCGTGCAGATGGACAGCAC
CGCTGAGC
AATGGAAGCGGGTAGGCCTTTGGGGCAGCGGCCAATAGCAG
CTTTGCTCC
TTCGCTTTCTGGGCTCAGAGGCTGGGAAGGGGTGGGTCCGGG
GGCGGGCT
CAGGGGGGGGCTCAGGGGCGGGGGGGGCGCCCGAAGGTCCT
CCGGAGGC
CCGGCATTCTGCACGCTTCAAAAGCGCACGTCTGCCGCGCTG
TTCTCCTCT
TCCTCATCTCCGGGCCTTTCGACCTGCAGCCCAAGCTTGGCA
ATCCGGTAC
TGTTGGTAAAGCCACCATGGAAGATGCCAAAAACATTAAGA
AGGGCCCAG
CGCCATTCTACCCACTCGAAGACGGGACCGCCGGCGAGCAGC
TGCACAAA
GCCATGAAGCGCTACGCCCTGGTGCCCGGCACCATCGCCTTT
ACCGACGCA
CATATCGAGGTGGACATTACCTACGCCGAGTACTTCGAGATG
AGCGTTCGG
CTGGCAGAAGCTATGAAGCGCTATGGGCTGAATACAAACCAT
CGGATCGTG
GTGTGCAGCGAGAATAGCTTGCAGTTCTTCATGCCCGTGTTG
GGTGCCCTG
TTCATCGGTGTGGCTGTGGCCCCAGCTAACGACATCTACAAC
GAGCGCGA
GCTGCTGAACAGCATGGGCATCAGCCAGCCCACCGTCGTATT
CGTGAGCA
AGAAAGGGCTGCAAAAGATCCTCAACGTGCAAAAGAAGCTA
CCGATCATA
CAAAAGATCATCATCATGGATAGCAAGACCGACTACCAGGG
CTTCCAAAG
CATGTACACCTTCGTGACTTCCCATTTGCCACCCGGCTTCAAC
GAGTACGA
CTTCGTGCCCGAGAGCTTCGACCGGGACAAAACCATCGCCCT
GATCATGA
ACAGTAGTGGCAGTACCGGATTGCCCAAGGGCGTAGCCCTAC
CGCACCGC
ACCGCTTGTGTCCGATTCAGTCATGCCCGCGACCCCATCTTCG
GCAACCAG
ATCATCCCCGACACCGCTATCCTCAGCGTGGTGCCATTTCACC
ACGGCTTC
GGCATGTTCACCACGCTGGGCTACTTGATCTGCGGCTTTCGG
GTCGTGCTC
ATGTACCGCTTCGAGGAGGAGCTATTCTTGCGCAGCTTGCAA
GACTATAAG
ATTCAATCTGCCCTGCTGGTGCCCACACTATTTAGCTTCTTCG
CTAAGAGCA
CTCTCATCGACAAGTACGACCTAAGCAACTTGCACGAGATCG
CCAGCGGC
GGGGCGCCGCTCAGCAAGGAGGTAGGTGAGGCCGTGGCCAA
ACGCTTCC
ACCTACCAGGCATCCGCCAGGGCTACGGCCTGACAGAAACA
ACCAGCGCC
ATTCTGATCACCCCCGAAGGGGACGACAAGCCTGGCGCAGTA
GGCAAGGT
GGTGCCCTTCTTCGAGGCTAAGGTGGTGGACTTGGACACCGG
TAAGACAC
TGGGTGTGAACCAGCGCGGCGAGCTGTGCGTCCGTGGCCCCA
TGATCATG
AGCGGCTACGTTAACAACCCCGAGGCTACAAACGCTCTCATC
GACAAGGA
CGGCTGGCTGCACAGCGGCGACATCGCCTACTGGGACGAGG
ACGAGCACT
TCTTCATCGTGGACCGGCTGAAGAGCCTGATCAAATACAAGG
GCTACCAG
GTAGCCCCAGCCGAACTGGAGAGCATCCTGCTGCAACACCCC
AACATCTT
CGACGCCGGGGTCGCCGGCCTGCCCGACGACGATGCCGGCG
AGCTGCCCG
CCGCAGTCGTCGTGCTGGAACACGGTAAAACCATGACCGAG
AAGGAGATC
GTGGACTATGTGGCCAGCCAGGTTACAACCGCCAAGAAGCTG
CGCGGTGG
TGTTGTGTTCGTGGACGAGGTGCCTAAAGGACTGACCGGCAA
GTTGGACG
CCCGCAAGATCCGCGAGATTCTCATTAAGGCCAAGAAGGGC
GGCAAGATC
GCCGTGTAATTCTAGTTGTTTAAACGAGCTCGCTAGCCTCGA
GTCTAGAGT
CGACCTGCAGG.

1.5 Dual-Luciferase Reporter Gene Plasmid Transfection and Detection of Activity

### 1) Cell Seeding and Culture

(1) Cells were cultured to a density of about 90% and washed twice with PBS.

(2) The supernatant was discarded, and an appropriate volume of 0.25% trypsin was added to digest the cells; after the cells became round, complete culture medium was added to terminate the reaction.

(3) Cells were blown using a 5-mL pipette tip, and the mixed solution was collected into a 15-mL test tube, then centrifuged at 150 g for 3 minutes.

(4) The supernatant was discarded and cells were counted. Cells were inoculated into 12-well culture plate according to the experimental requirement, and the culture plate was placed in an incubator at 37° C. and 5% CO2 for culture overnight.

(5) After 24 h, transfection treatment was performed according to the experimental requirements.

### 2) Cell Transfection

(1) Transfection was performed when the cell density was about 70%.

(2) The transfection reagents, solution 1 and solution 2 were prepared, taking three wells in a 12-well plate as a group as an example below:

Solution 1:150 μL of optimization olution+9 μL of liposome 3000.

Solution 2:150 μL of optimization olution+1.5 μg of plasmid+6 μL of P3000+45 μmol of fragment.

(3) The solution 1 and solution 2 were mixed and placed at room temperature for 15 minutes.

(4) The mixture of olution 1 and solution 2 was added dropwise into the wells, then the culture plate was shaken to mix gently, and continued to culture at 37° C. and 5% CO2 for 24 hours.

### 3) Detection of Luciferase Activity

(1) After cell culture medium was aspired completely, cells were rinsed twice with 1×PBS and 250 μL of cell lysis solution was added.

(2) After the sample, firefly luciferase detection reagent and Renilla luciferase detection reagent were equilibrated to room temperature, the following steps were carried out:

(3) 20 μL of cell lysis sample was added to an ELISA plate.

(4) 100 μL of firefly luciferase detection reagent was added to each well, gently blown and mixed evenly using a pipette, then the result was read.

(5) 100 μL of Renilla luciferase detection reagent was added to each well, gently shaken and mixed evenly using an ELISA reader, then the result was read.

The luciferase ratio was calculated according to the results.

### 2. Experimental Results

2.1 miR-22-3p Directly Targeted MEG8 and RTL1

MEG8 could affect the proliferation and differentiation of yak myoblasts, which was consistent with the results predicted in the lncRNA measured in the early stage. In addition, a ceRNA regulatory mechanism worthy of attention was found in the MvsE and AvsE groups. Therefore, it was speculated in the present disclosure that a MEG8-RTL1-miR22 regulatory network existed in myoblasts and affected the differentiation of myoblasts.

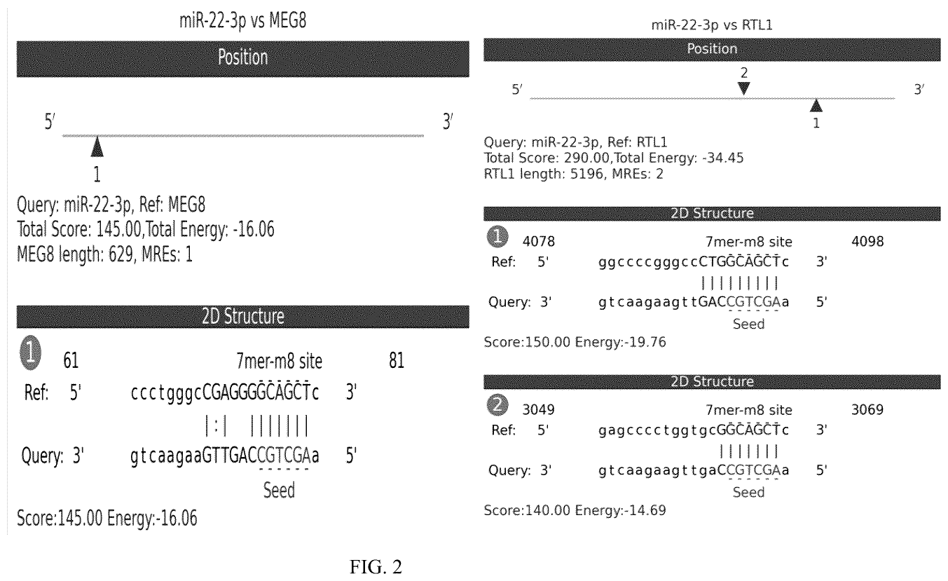

The differential lncRNA analysis and differential mRNA-lncRNA cis/trans network results showed that, RTL1 had a cis-regulatory target gene-MEG8 (XR_314844.1) in the two comparison groups. In the present disclosure, in order to further explore the interaction between RTL1-MEG8, using MEG8 as the base point, bioinformatics analysis was performed to screen out the differentially expressed novel211_mature; and bta-miR-22-3p was a potential target of MEG8/RTL1. Meanwhile, the online software TargetScan and RNAhybrid were used to jointly predict that there were many target miRNAs for MEG8/RTL1, among which miR-22-3p attracted attention. There was an identical complementary base pairing binding DNA sequence among MEG8, miR-22-3p and miR-22-3p target gene RTL1 (FIG. 2).

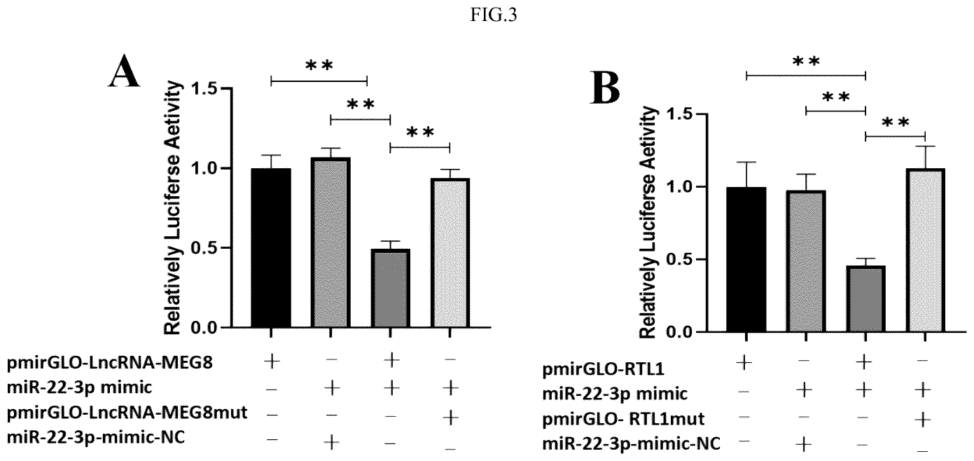

In this embodiment, to further verify the regulatory relationship among miR-22-3p and MEG8 and RTL1, the binding sites of miR-22-3p on RTL1 and MEG8 genes were synthesized through base synthesis, and wild-type (unmutated binding site) and mutant (site-directed mutation of binding site) dual-luciferase reporter gene vectors were constructed respectively. Meanwhile, General Biol (Anhui) Co., Ltd. synthesized miR-22-3p mutant mimics, and the interaction between miR-22-3p and MEG8 and RTL1 was detected by dual luciferin experiment. The results showed that miR-22-3pmimic could significantly reduce the activity of wild-type RTL1 dual-luciferase reporter gene vector (MEG8-pmirGLO), while miR-22-3p mimic had no effect on mutant MEG8 dual-luciferase reporter gene vector (MEG8-mut-pmirGLO). When miR-22-3p mimic was mutated, it had no effect on the wild-type or mutant MEG8 vector (A in FIG. 3). Similarly, miR-22-3p mimic could also significantly reduce the activity of wild-type RTL1 dual-luciferase reporter gene vector (RTL1-pmirGLO), while miR-22-3p mimic had no effect on mutant RTL1 dual-luciferase reporter gene vector (RTL1-mut-pmirGLO). When miR-22-3p mimic was mutated, it had no effect on the wild-type or mutant RTL1 vectors (B in FIG. 3). The above results showed that there was a direct targeting relationship between MEG8 and miR-22-3p, and between miR-22-3p and RTL1.

2.2 miR-22-3p Promoted Myoblast Differentiation

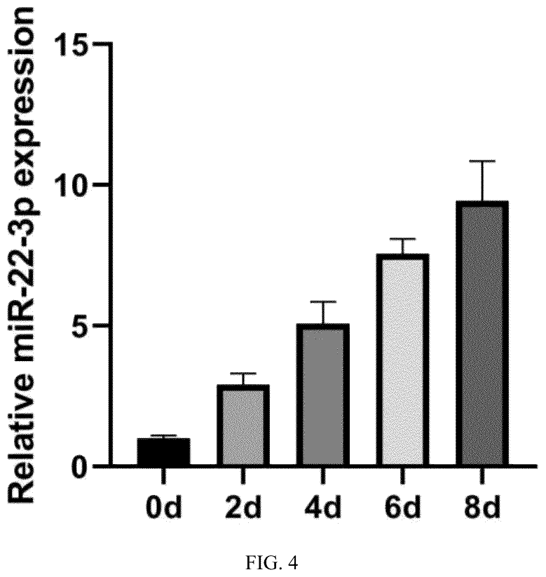

Based on the above results, miR-22-3p directly targeted MEG8 and RTL1, indicating that there was a direct regulatory relationship between them. Therefore, the role of miR-22-3p in the satellite cell differentiation process was further studied in this example. The expression level of miR-22-2p was detected at different time points of myoblast differentiation. The results showed that miR-22-3p continued to rise during the differentiation process (FIG. 4).

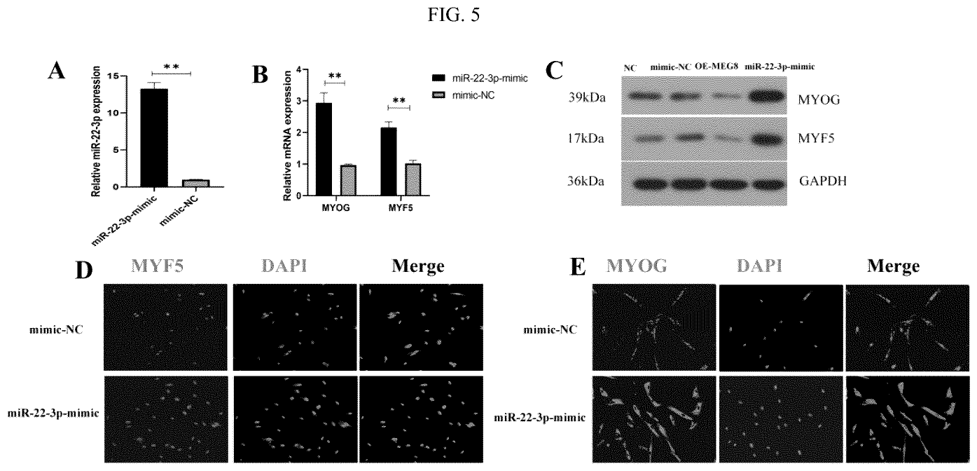

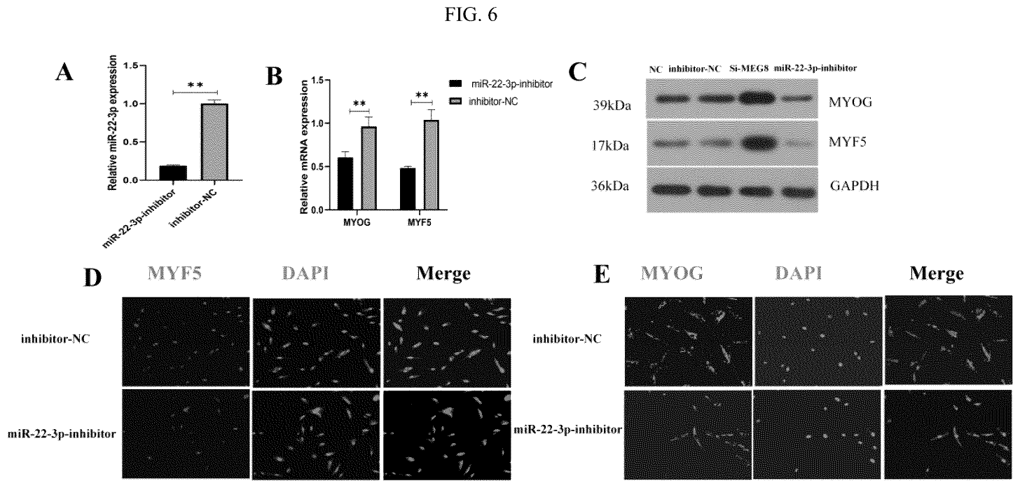

In this example, to further study the role of miR-22-3p in myoblast differentiation process, miR-22-3p mimic and miR-22-3p inhibitor were used to transfect myoblasts respectively; after transfection, cells were induced to differentiate, and 24 hours after induction of differentiation, cells were collected to detect mRNA and protein levels. QPCR and Western blot results showed that miR-22-3pmimic significantly promoted the expression of miR-22-3p, and significantly increased the expression of differentiation marker genes MYF5, MYOG at the mRNA and protein levels (FIG. 5). MiR-22-3pinhibitor significantly inhibited the expression of miR-22-3p, and significantly promoted the expression of MYF5, MYOG at the mRNA and protein levels (FIG. 6). In the present disclosure, immunofluorescence experiments were conducted to detect the effects of miR-22-3p mimic and inhibitor on myotube formation. The results showed that miR-22-3p mimic significantly promoted the formation of myotubes (D, F in FIG. 5), while miR-22-3p inhibitor significantly inhibited the formation of myotubes (D, F in FIG. 6). The above results indicated that miR-22-3p had a promoting effect in the differentiation process of myoblasts.

### 2.3 RTL1 Inhibited Myoblast Differentiation

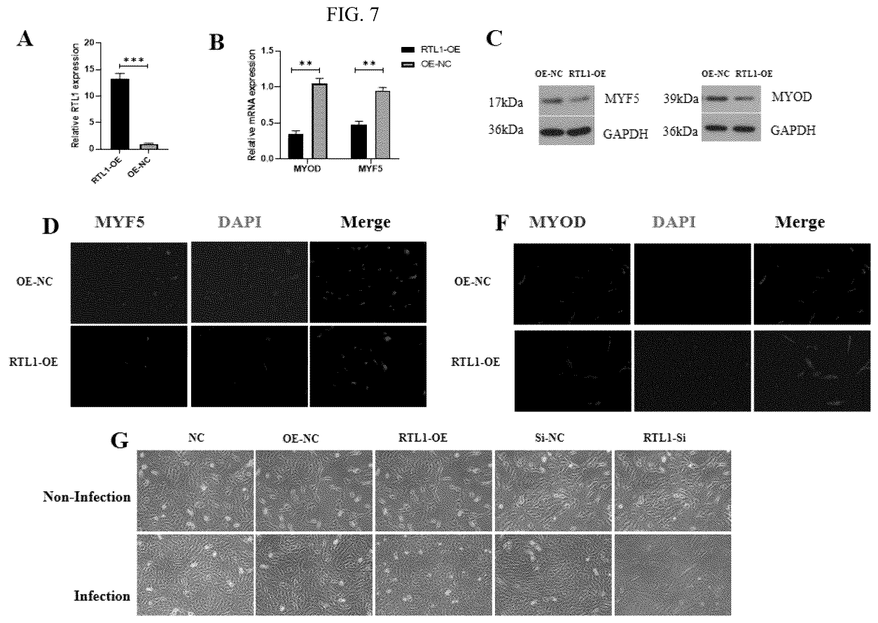

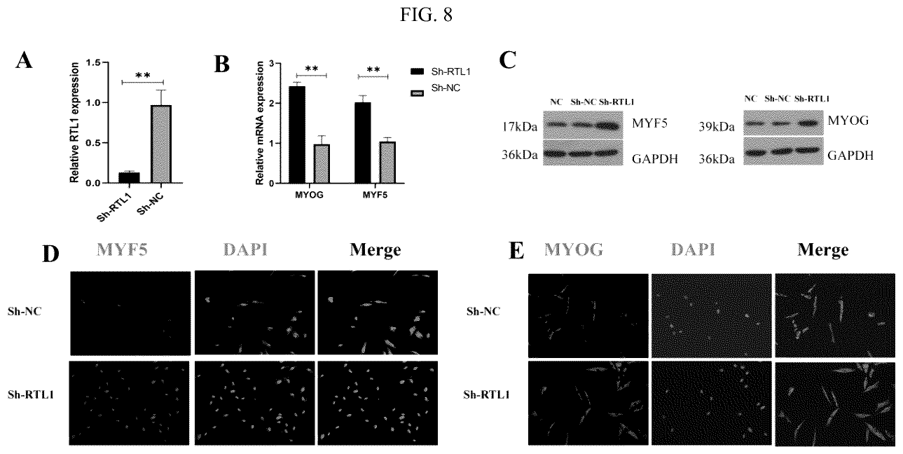

In this example, RTL1-Si and RTL-OE overexpression vectors were used to transfect myoblasts for further experiments. The transfection procedure was same as that mentioned above; 24 hours after transfection, cells were collected for detection. The results showed that RTL-OE significantly promoted the expression of RTL1, and significantly reduced the expression of MYOG and MYF5 at the mRNA and protein levels (FIG. 7), and had a significant effect on the protein and mRNA expression levels of MYOG and MYF5. RTL1-Si significantly reduced the expression of RTL1, and significantly reduced the expression of differentiation marker genes MYOG and MYF5 at the mRNA and protein levels (FIG. 7). In the present disclosure, immunofluorescence experiments were performed to detect the effects of RTL1-Si and RTL-OE on myotube formation. The results showed that RTL-OE significantly inhibited the formation of myotubes (D in FIG. 7), while RTL-Si significantly promoted the formation of myotubes (G in FIG. 7, FIG. 8). The above experimental results indicated that RTL1 inhibited myoblast differentiation.

2.4 MEG8 Affected Myoblast Differentiation by Competitively Binding to miR-22-3p with RTL1

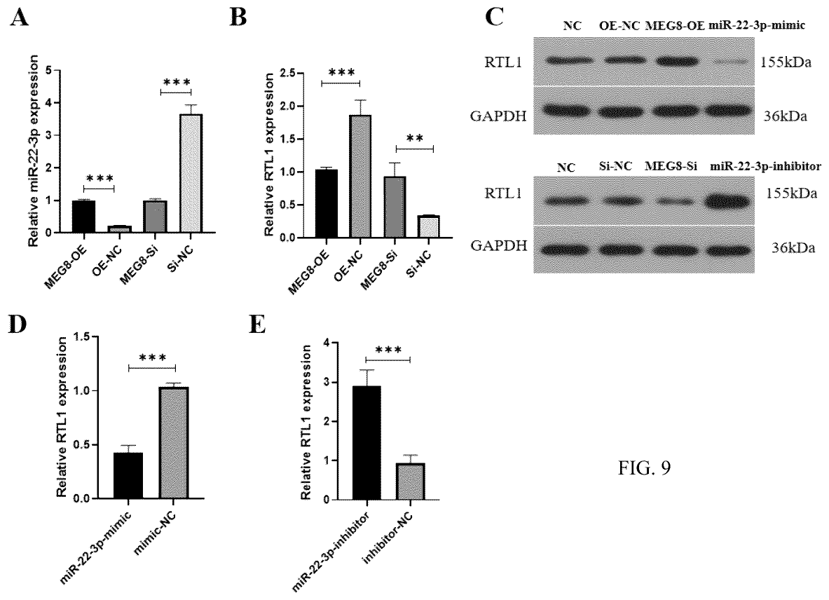

In this example, in order to further explore the relationship between MEG8, miR-22-3p, and RTL1, the mutual regulatory relationship between MEG8, miR-22-3p, and RTL1 was detected at first. The results showed that overexpression of MEG8 significantly up-regulated the expression of miR-22-3p, and significantly down-regulated the expression of RTL1 at the mRNA and protein levels. Knockdown of MEG8 significantly down-regulated the expression of miR-22-3p (AC in FIG. 9), and significantly up-regulated the expression of RTL1 at the mRNA and protein levels (BC in FIG. 9). While miR-22-3pmimic significantly inhibited the expression of RTL1 (CD in FIG. 9), on the contrary, miR-22-3p inhibitor significantly promoted the expression of RTL1 (CE in FIG. 9). The results indicated the positive regulatory relationship between MEG8 and RTL1 and the negative regulatory relationship between miR-22-3p and RTL1.

Although the present disclosure has been described in detail in conjunction with the foregoing embodiments, these embodiments are only a part of embodiments of the present disclosure, and are not all of embodiments thereof. All other embodiments obtained by persons of ordinary skill in the art based on the embodiments of the present disclosure without creative efforts shall fall within the protection scope of the present disclosure.

What is claimed is:

1. Use of miR-22-3p in promoting differentiation of yak myoblasts for non-disease treatment purposes.

2. Use of miR-22-3p in promoting formation of yak myotubes for non-disease treatment purposes.


SEQUENCE LISTING (2 sequences)

SEQ ID NO: 1 - DNA, 156 bp, synthetic construct
  source 1..156: mol_type = other DNA
actaaagatatttgcatgtcgctatgtgttctgggaaatcaccataaacgtgaaatgtctttggatttgggaatcttataagttctgtatgagaccactcagacgatgatggagcgtaagattcaagagatcttacgctccatcatcgtctttttt

SEQ ID NO: 2 - DNA, 7350 bp, synthetic construct
  source 1..7350: mol_type = other DNA
catgcaagctgatccggctgctaacaaagcccgaaaggaagctgagttggctgctgccaccgctgagcaataactagcataaccccttggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggtttttttaagcaagtaaaacctctacaaatgtggtaaaatcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaaccaaaggctggagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagaccggtggtgggagcggaggtggcggatcaggtggcggaggctccggagggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatcctctttgcgcttgcgttttcccttgtccagatagcccagtagctgacattcatccggggtcagcaccgtttctgcggactggctttctacgtaatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttcaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattcgaaatgaccgaccaagcgacgcccaaccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattctagttgtttaaacgagctcgctagcctcgagtctagagtcgacctgcagg